(19) United States
(12) Patent Application Publication
(10) Pub. No.: US 2003/0049800 A1
(43) Pub. Date: Mar. 13, 2003

(54) Compositions and methods for producing antibodies to low molecular weight analytes

(76) Inventors: Calvin A. Saravis, Glencoe, IL (US); Richard B. Cook, Chelmsford, MA (US)

Correspondence Address: FISH & NEAVE, 1251 AVENUE OF THE AMERICAS, 50TH FLOOR, NEW YORK, NY, 10020-1105, US

(21) Appl. No.: 10/015,833

(22) Filed: Dec. 12, 2001

Related U.S. Application Data
(60) Provisional application No. 60/256,180, filed on Dec. 15, 2000.

(51) Int. Cl. A61K 39/00; C12P 21/04; C08B 37/04

(52) U.S. Cl. 435/70.21; 424/184.1; 435/326; 534/16; 536/3

(57) ABSTRACT

Improved immunogenic compositions and methods for producing antibodies to low molecular weight analytes are presented. In one series of embodiments particularly useful for production of antibodies having specificity for metal ions, nonimmunogenic polymers having natural metal ion-binding activity, typically alginates, are used, after contact with metal ions, as an immunogenic composition. In another series of embodiments, immunogens are enmeshed in a crosslinked protein network to create a potent immunogenic composition. In the latter embodiments, gelatins of cold-water fish prove surprisingly efficacious.

### CROSS-REFERENCE TO RELATED APPLICATIONS

This application claim priority to U.S. provisional application serial No. 60/256,180 filed on Dec. 15, 2000, the disclosure of which is incorporated herein by reference in its entirety.

### FIELD OF THE INVENTION

This invention relates to improved methods for producing antibodies, particularly polyclonal antibodies with specificity for low molecular weight analytes, such as heavy metal ions.

### BACKGROUND OF THE INVENTION

The mammalian humoral immune system has for decades been exploited to generate analytical reagents of high affinity and specificity. Antibody reagents have been made to an extraordinary variety of molecules of scientific and clinical interest, from proteins to carbohydrates, lipids to nucleic acids, synthetic peptides to synthetic organic molecules. Linscott's Directory (Linscott's Directory, 4877 Grange Road, Santa Rosa, Calif. USA) lists nearly 100,000 such reagents that are readily available for purchase; no doubt tens of thousands, perhaps hundreds of thousands, of others can be found in research laboratories around the world.

Some molecules, such as large proteins, prove highly immunogenic. Others prove less immunogenic, and thus more recalcitrant to production of specific antibodies.

It has long been known, for example, that small molecules often do not themselves trigger a humoral immune response. Long before the phenomenon was understood at the cellular level, it had been shown that conjugation of such small haptens to carrier proteins allowed the production of anti-hapten antibodies.

It has also long been known that certain chemical classes of molecules, irrespective of their size, are less readily recognized by the mammalian immune system. Lipids and nucleic acids, for example, typically prove to be poor immunogens, although they can often be rendered immunogenic when conjugated to proteins.

Among small analytes for which specific antibodies have long been desired are metal ions.

Metals are significant environmental contaminants, and can pose risk even at extremely low levels. The U.S. Environmental Protection Agency has, for example, just reduced the acceptable level of arsenic in drinking water from 50 to 10 parts per billion (ppb). Environmental monitoring and remediation programs thus require reagents and methods that are at once highly sensitive and extremely specific. Antibodies with specificity for metal ions could in theory meet those demands.

Metals are also a significant cause of human morbidity and mortality. Indeed, several states require that children be tested for blood lead levels before school entry. Clinical monitoring and diagnostic efforts thus require reagents and methods that are at once highly sensitive and extremely specific. Again, antibodies with specificity for metal ions could in theory meet these demands.

However, metal ions are considered too small alone to elicit a humoral response, and when presented in elemental form metals are poorly recognized by the mammalian humoral immune system. Furthermore, many of the metal ions for which specific antibodies are desired are toxic to the immunized host, interfering with antibody production.

One solution to these problems has been to complex the metal ion to a larger, immunogenic, carrier. Typically, this has been done by binding the metal ion to one or more chelating ligands which, in turn, are covalently linked to an immunogenic carrier.

For example, U.S. Pat. No. 4,722,892 describes covalent linkage of aminobenzyl-EDTA, a chelator, to an immunogenic carrier. The carrier is typically a protein, such as keyhole limpet hemocyanin (KLH). The immunogenic chelating complex is charged with metal ion and then used as an immunogen to generate monoclonal antibodies that are specific for the metal ion as chelated by EDTA.

U.S. Pat. Nos. 5,908,790 and 5,907,034 describe immunogens comprising yttrium ion-charged EDTA (or DTPA) covalently linked to carrier proteins. In contrast to U.S. Pat. No. 4,722,892, the stated goal is to obtain antibodies that bind specifically to the chelator, whether or not complexed to a metal ion.

U.S. Pat. No. 5,476,939 describes the synthesis of tridentate chelators that can be covalently linked to an immunogenic carrier. Loaded with metal ion, the chelator-carrier complex is capable of eliciting antibodies that bind specifically to the metal ion in its chelated form. The chelating ligands are specifically chosen to form highly stable ligand-metal linkages, in order to forestall toxicity caused by release of free metal ion during the months' long immunization protocol. The carrier can be a protein, such as BSA, keyhole limpet hemocyanin (KLH), thyroglobulin, even immunoglobulin, or can be a carbohydrate, polysaccharide, lipopolysaccharide, poly(amino)acid, or nucleic acid.

U.S. Pat. Nos. 6,111,079, 5,972,656, and 5,639,624, and 5,503,987 describe the coordination of metal ions to the end of a biopolymer spacer arm that is covalently bonded to an immunogenic carrier. The spacer arm can be an oligopeptide, such as glutathione, an aliphatic compound or an aliphatic fragment. The spacer arm is said to be semi-rigid and to hold the small moiety in an exposed position relative to the carrier. The carrier is itself a biopolymer such as a protein, a polysaccharide, or polyamide. With metal ions bound, the complex is suitable for production of monoclonal antibodies that are specific for the metal ion as coordinated by the spacer arm.

Other examples in which antibodies have been generated against metal ion complexes with EDTA and other small, non-peptide, chelators have been described. Love et al., Biochemistry 32:10950-10959 (1993); Reardan et al., Nature 316:265 (1985); Boden et al., Bioconjugate Chem. 6:373-379 (1995); Blake et al., J. Biol. Chem. 271:27677-27685 (1996); Khosraviani et al., Bioconjugate Chem. 11:267-277 (2000).

In an alternative to use of a chelating ligand covalently bound to a carrier, U.S. Pat. Nos. 5,532,136 and 5,620,856 describe an immunogenic complex in which a metal ion is bound directly to a naturally-occurring polypeptide that has intrinsic metal-binding affinity, such as δ-aminolevulinic acid dehydratase (ALAD)

In each of the approaches described above, the carrier is itself immunogenic, and thus elicits antibodies that are specific for the carrier itself.

Where the antibodies are desired to be monoclonal, this concomitant immune response to carrier obligates additional screening of the resultant hybridomas to eliminate those that secrete antibodies that recognize (i.e., bind specifically to) epitopes contributed by the carrier.

Where the antibodies are desired to be polyclonal, concomitant production of antibodies specific for carrier epitopes presents analogous difficulties.

Typically, polyclonal antibodies are affinity purified from serum using the immunogen as an affinity moiety. Where antibodies are, however, additionally produced at high titers to a carrier present in the immunogen, the carrier must typically be used in an additional, negative, affinity selection (absorption). Each of these affinity purification cycles presents opportunities for contamination of the antibodies, for example by leaching of the selecting affinity moiety into the purified antibody pool. Each of the affinity purification cycles also risks degradation of the antibody pool, due in part to the harshness of elution conditions. Adding a negative selection compounds these problems.

There thus exists a need in the art for compositions and methods that at once permit small molecules, such as metal ions, to be rendered suitably immunogenic as to elicit antibodies, but that do not at the same time elicit a significant humoral immune response to a conjugated carrier. There exists a particular need for compositions and methods that allow high titers of polyclonal antibodies to be produced to small molecules, such as metal ions, without producing high titers of antibodies to a carrier component of the immunogen.

### SUMMARY OF THE INVENTION

The present invention solves these and other problems in the art by providing, in a first aspect, an immunogenic composition capable of eliciting high titer polyclonal antibodies to metal ion chelates and free metal ions.

The present inventors have discovered that nonimmunogenic polymers having natural metal complexing activity, including various polysaccharides such as alginates, can be used directly as immunogens; after binding of metal ions, these naturally-chelating nonimmunogenic polymers, without the further addition of a carrier protein, are capable of eliciting high titers of polyclonal antibodies having specificity for the complexed metal ion, and do so without provoking significant production of antibodies to the polymer itself.

Accordingly, the immunogenic composition of this aspect of the invention comprises a naturally-chelating nonimmunogenic polymer, a metal ion, and an adjuvant, wherein the metal ion is bound to the naturally-chelating nonimmunogenic polymer.

In certain preferred embodiments, the naturally-chelating nonimmunogenic polymer is an alginate; the metal ion is selected from the group consisting of ionic lead, mercury, cadmium, aluminum, lithium, strontium, copper, aluminum, iron, antimony, arsenic, bismuth, chromium, copper, molybdenum, nickel, thallium, technetium, gadolinium, barium, indium, and tin, and the adjuvant is selected from the group consisting of complete Freund's adjuvant (CFA), incomplete Freund's Adjuvant (IFA), montamide ISA (Incomplete Seppic Adjuvant), Ribi Adjuvant System (RAS); TiterMax; Syntex Adjuvant Formulation (SAF); aluminum salts; nitrocellulose-adsorbed antigen; immune-stimulating complexes (ISCOMs); and Gerbu adjuvant.

The naturally-chelating nonimmunogenic polymer is typically particulated, e.g. formed as a composition of beads, and can further comprise thermally gelling polymers such as agarose.

The immunogenic compositions of this aspect of the invention can further comprise a nonpolysaccharide chelator capable of chelating the same metal ion as is bound to the naturally-chelating nonimmunogenic polymer. The chelator can be selected from the group consisting of EDTA, DTPA, meso-2,3-dimercapto succinic acid (DMSA), 2,3-dimercapto-1-propane sulfonate (DMPS), dimercaptopropanol, metallothionein, lactate, penicillamine, deferoxamine, and triethylene tetramine dihydrochloride, and is often EDTA.

The present inventors have further discovered that crosslinking certain poorly immunogenic proteins in the presence of an immunogen, thus enmeshing the immunogen within a macromolecular protein scaffold, creates an immunogenic composition that evokes a robust humoral immune response to the enmeshed immunogen, but without producing significant titers of antibodies to the crosslinked protein.

Accordingly, the immunogenic compositions can further comprise a crosslinked protein, wherein the protein has been crosslinked in the presence of the naturally-chelating nonimmunogenic polymer and the nonpolysaccharide chelator.

The enmeshing protein can be selected from a number of proteins. Particularly useful among such enmeshing proteins are gelatins that are liquid at room temperature, with gelatins from cold-water fish presenting surprising advantages. Typically, the protein, often cold-water fish gelatin, is present in an amount sufficient, upon crosslinking, to create a gel at room temperature.

In a second aspect, the invention provides immunogenic compositions that comprise a particulate naturally-chelating nonimmunogenic polymer complexed with a metal ion, and an adjuvant. The particulate, metal ion-complexed naturally-chelating nonimmunogenic polymer in certain embodiments is depotized, and the metal ion of the depotized particulate metal ion-complexed naturally-chelating nonimmunogenic polymer is dynamically bound by a plurality of chelators.

The advantages of using crosslinked cold-water fish gelatin to enmesh an immunogen transcend its use with naturally-chelating nonimmunogenic polymer/metal ion complexes.

Accordingly, it is another aspect of the invention to provide immunogenic compositions in which the enmeshed immunogen is not limited to chelates of nonimmunogenic polymers and metal ions.

The immunogenic composition of this aspect of the invention comprises an immunogen and a crosslinked gelatin soluble at room temperature, wherein the gelatin is crosslinked in the presence of the immunogen. In preferred embodiments, the gelatin is a cold-water fish gelatin.

The immunogenic compositions of this aspect of the invention can further comprise an adjuvant.

In another aspect, the invention provides a method of making an antibody reagent, the method comprising immunizing a nonhuman animal with the immunogenic compositions of the invention, and then isolating an antibody that binds to the immunogenic composition.

The antibody can be isolated directly from the serum of the immunized nonhuman animal, or can be obtained by first isolating a cell that secretes an antibody that binds to the immunogenic composition. In typical embodiments of this latter approach, the cell is a clonal cell line. The method can optionally further comprise affinity purifying the desired antibody reagent.

In another aspect, the invention provides an antibody reagent, comprising at least one isolated antibody, wherein the antibody is specific for a metal ion as complexed to a nonimmunogenic polymer. In useful embodiments, the nonimmunogenic polymer is a polysaccharide such as an alginate, and the metal ion is selected from the group consisting of ionic lead, mercury, cadmium, aluminum, lithium, strontium, copper, aluminum, iron, antimony, arsenic, bismuth, chromium, copper, molybdenum, nickel, thallium, technetium, gadolinium, barium, indium, and tin.

### BRIEF DESCRIPTION OF THE DRAWINGS

The above and other objects and advantages of the present invention will be apparent upon consideration of the following detailed description taken in conjunction with the accompanying drawings, in which like characters refer to like parts throughout, and in which:

FIG. 1 is a perspective view of a device in which antibodies with specificity for metal ion chelates are used to detect and quantify metal ions that have been drawn from a body fluid by transdermal extraction into a hydrogel.

### DETAILED DESCRIPTION OF THE INVENTION

The present inventors have discovered that nonimmunogenic polymers having natural metal ion complexing activity, such as alginates, can be used directly as immunogens; after binding of metal ions, these nonimmunogenic polymers, without the further addition of a carrier protein, are capable of eliciting high titers of polyclonal antibodies having specificity for the complexed metal ion, and do so without provoking significant production of antibodies to the polymer itself. The present inventors have further discovered that crosslinking certain poorly immunogenic proteins in the presence of an immunogen, thus enmeshing the immunogen within a macromolecular protein scaffold, creates an immunogenic composition that evokes a robust humoral immune response to the enmeshed immunogen, but without producing significant titers of antibodies to the crosslinked protein.

Separately and together, these discoveries permit the rapid, cost-effective generation of antibodies to small molecular weight analytes, particularly metal ions, without the confounding presence of antibodies to a carrier. In many applications, including diagnostic applications, polyclonal antibodies produced using the compositions and methods of the present invention can be used directly, without affinity purification.

Definitions

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513), the disclosure of which is incorporated herein by reference in its entirety).

As used herein, “antigen” refers to a ligand that can be bound by an antibody; an antigen need not itself be immunogenic. The portions of the antigen that make contact with the antibody are denominated “epitopes”.

“Specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 10⁻⁷ M, with specific binding reactions of greater specificity typically having affinity or avidity of at least 10⁻⁸ M to at least about 10⁻¹⁰ M.

Compositions and Methods

In a first aspect, the invention provides an immunogenic composition that comprises a naturally-chelating nonimmunogenic polymer, a metal ion bound thereto, and an adjuvant. These compositions are useful for producing antibodies with specificity for the metal ion, both as complexed and as a free ion.

The naturally-chelating nonimmunogenic polymer is typically a gelled or microparticulated polycarboxylated polymer. It is most typically a polysaccharide.

A number of polysaccharide compositions have been developed that have been engineered specifically to bind metal ions with high affinity. Among these are agarose beads derivatized with nitrilo triacetic acid (NTA) and imino diacetic acid (IDA). Originally developed to chelate metal ions in a form useful for purifying polyhistidine-tagged fusion proteins, these chelating polysaccharide gels can readily be used in the immunogenic compositions of the present invention.

Typically, however, these compositions are sold prior-chelated with metal ions, typically nickel or cobalt ions, and thus require exchange of metal ions to produce immunogens capable of presenting other metal ions to the immune system. Even were such exchange efficient, the cost in many cases would be high.

The naturally-chelating nonimmunogenic polymer of the immunogenic compositions of the present invention can thus usefully be selected from polysaccharides that are not prior-chelated with metal ions. Among such polysaccharides, those having natural metal ion-chelating activity prove particularly useful.

Alginates, cell-wall constituents of brown algae (Phaeophyceae, mainly Laminaria), are known to complex atomically heavy metal ions with graded affinity: lead and other heavy metal ions are taken up in preference to sodium, potassium, and other metal ions of lower atomic number. Alginates thus prove particularly useful in the immunogenic compositions of the present invention.

Alginates are linear unbranched polymers containing β-(1→4)-linked D-mannuronic acid (M) and α-(1→4)-linked L-guluronic acid (G) residues.

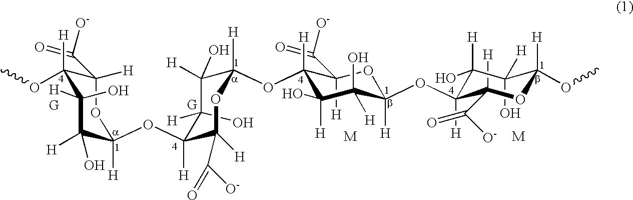

Alginates are not random copolymers but, according to the source algae, consist of blocks of similar and strictly alternating residues (i.e. MMMMMM, GGGGGG and GMGMGMGM), each of which have different conformational preferences and behavior.

Alginates form thermally stable, cold-setting gels upon addition of cations, with gelation depending on the ion (Mg²⁺<<Ca²⁺<Sr²⁺<Ba²⁺), on the relative G/M content of the polymer, and on the average chain length.

For use in the present invention, the alginate gel can be cast or polymerized in any shape and any convenient size. Usefully, the alginate is cast or polymerized in a form having a high surface to volume ratio, thus exposing as many metal ion-complexing sites as possible for a given volume of gel. Typically, the alginate is particulated.

The alginate can thus usefully take the form of beads, with a mean diameter of at least about 1 μm, often at least about 5 μm, typically at least about 10 μm, 20 μm, 25 μm, even 50 μm, 75 μm, 100 μm or more. The beads will typically have a mean diameter of no more than about 2000 μm, often no more than about 1000 μm, 750 μm, even no more than about 500 μm, 400 μm, 300 μm, or even no more than about 250 μm, with beads of about 50 μm to 250 μm being typical. Alginate microspheres can have average diameters of 2000, 3000, or even 4000 μm, although smaller diameters are often preferred.

Alginate beads have typically been produced by dripping alginate solution into a CaCl₂ bath. More recently, techniques have been described that permit inclusion of alginate in spheres of thermally gelling polymers, such as agarose. See, e.g., U.S. Pat. No. 6,248,268 and WO 00/29466, the disclosures of which are incorporated herein by reference in their entireties. Alginate extracts and alginate beads are also readily available commercially (e.g., from FMC BioPolymer, Philadelphia, Pa., USA; Hallcrest, Inc., Glenview, Ill. USA; International Specialty Products, Wayne, N.J., USA).

In the compositions of the present invention, the metal of the immunogenic composition is chosen based upon the desired specificity of the antibodies.

Heavy metal ions that can be used include those for which monitoring human exposure is clinically important, such as ionic lead, mercury, and cadmium. Other metal ions that usefully can be included include ionic strontium, lithium, copper, aluminum, iron, antimony, arsenic, bismuth, chromium, copper, molybdenum, nickel, thallium, technetium, gadolinium, yttrium, and tin. Particularly useful immunogens are those that include lead or mercury ions.

The metal ion is complexed noncovalently, often reversibly, to the nonimmunogenic polymer. Without wishing to be bound by theory, it is believed that the metal ions are chelated by chemical groups naturally present within the nonimmunogenic polymer, and that the binding is saturable. Accordingly, the nonimmunogenic polymer component of the immunogenic composition is at times referred to herein as the “polymer chelator” and the binding of the metal as “chelation”, without intending thereby to be limited to chemical bonding mechanisms found in chelators such as EDTA.

The metal ion can be present in subsaturating or saturating amounts, and can be present in excess of the saturable binding sites of the polymer chelator.

The composition further comprises at least one adjuvant in an amount sufficient to augment antibody production in the immunized host.

Adjuvants are well known in the immunological arts, and need not here be described in detail. See, e.g., Bennett et al., “A comparison of commercially available adjuvants for use in research,” J. Immunological Methods, 153:31-40 (1992); Jennings, “Review of selected adjuvants used in antibody production,” ILAR Journal 37(3):119-125 (1995). See also Harlow et al. (eds.), Antibodies: A Laboratory Manual, Cold Spring Harbor Laboratory (1998) (ISBN: 0879693142); Coligan et al. (eds.), Current Protocols in Immunology, John Wiley & Sons, Inc. (2001) (ISBN: 0-471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907), the disclosures of which are incorporated herein by reference.

Briefly, the compositions of the present invention can include any known adjuvant, including complete Freund's adjuvant (CFA), incomplete Freund's Adjuvant (IFA), montamide ISA (Incomplete Seppic Adjuvant), Ribi Adjuvant System (RAS) (an oil-in-water emulsion that contains detoxified endotoxin and mycobacterial cell wall components in 2% squalene); TiterMax (a water-in-oil emulsion combining a synthetic adjuvant and microparticulate silica with the metabolizable oil squalene; the copolymer is the immunomodulator component; antigen is bound to the copolymer and presented to the immune cells in a highly concentrated form); Syntex Adjuvant Formulation (SAF) (a preformed oil-in-water emulsion that uses a block copolymer for a surfactant and a muramyl dipeptide derivative as the immunostimulatory component in squalene, a metabolizable oil); aluminum salts (such as aqueous aluminum magnesium hydroxide); nitrocellulose-adsorbed antigen; immune-stimulating complexes (ISCOMs) (antigen modified saponin/cholesterol micelles) (Quil A is one example, QS-21 is another); and Gerbu adjuvant (GERBU Biotechnik GmbH, Heidelberg, Germany) (an aqueous phase adjuvant that uses immunostimulators in combination with zinc proline).

In a typical immunization protocol, which can involve iterative immunizations over several months, the adjuvant chosen for inclusion in the immunogenic composition can, and indeed may desirably, be changed, with the polymer chelator and metal ion remaining the same. For example, initial immunizations can be performed using complete Freund's adjuvant in the immunogenic composition, with subsequent immunizations being performed using incomplete Freund's adjuvant or aluminum salts in the immunogenic composition.

To prepare the immunogenic composition, the metal is typically contacted to the polymer chelator as an ionic salt in aqueous solvent. For example, lead can usefully be in the form of lead acetate and mercury conveniently in the form of mercury acetate. The polymer chelator can be in the form of a gel, typically a gel bead, or can be gelled in the presence of the metal.

The adjuvant can be added directly thereafter. Typically, however, excess water is first removed, for example by lyophilization.

When dried before addition of adjuvant, the polymer chelator-metal ion composition is thereafter typically rendered particulate before addition of adjuvant. This helps ensure more even dispersion of the polymer chelator-metal ion immunogen within the liquid adjuvant. Uniformity is not required, however. If not first dried, the polymer chelator-metal ion composition is typically macerated or otherwise increased in surface area before addition of adjuvant.

The composition can optionally include other components.

Thus, as noted above, the polymer chelator can be included in gels that further comprise other polymers, such as thermally-gelling polymers.

For example, as set forth in the Examples herein below, the polymer chelator can be an alginate that is incorporated into an agarose gel bead, with or without crosslinking therebetween. Methods for making such alginate-containing agarose gel beads are described, inter alia, in U.S. Pat. No. 6,248,268 and WO 00/29466, the disclosures of which are incorporated herein by reference in their entireties. Crosslinking can be effected, e.g., using divinylsulfone or bisepoxides or the like.

As noted above, the metal ion can be present in excess of the number of metal ion binding sites presented by the polymer chelator. Free metal ions can be toxic, however—indeed potentially fatal—to the host animal, thus interfering with or preventing adequate antibody production.

Accordingly, the immunogenic composition of the present invention can further comprise a nonpolysaccharide chelator, often in quantity sufficient to bind any metal ions present in excess of metal ion binding sites of the polymer chelator.

The nonpolysaccharide chelator can include any known chelator capable of binding the metal ion included within the immunogenic composition. The chelator can, for example, be EDTA or the related molecule, DTPA (diethylenetriaminepentaacetic acid). The nonpolysaccharide chelator can belong to the group of dithiol group chelators, such as meso-2,3-dimercapto succinic acid (DMSA) (Succimer), 2,3-dimercapto-1-propane sulfonate (DMPS) (Dimaval, Unithiol), dimercaptopropanol (British anti-Lewisite (BAL), Dimercaprol). The nonpolysaccharide chelator can be metallothionein, lactate, penicillamine (for copper), deferoxamine (equivalently denominated “desferoxamine”; for iron), or triethylene tetramine dihydrochloride (Trien).

In addition to reducing toxicity, the nonpolysaccharide chelator can also serve to improve immunogenicity to metal ion immunogens by other mechanisms. Without intending to be bound by theory, the inventors believe that the nonpolysaccharide chelator facilitates presentation of the metal ion to the host immune system in forms additional to those presented by the polymer chelator, improving immunogenicity. The effect is believed to occur whether or not excess metal ion is present, in part due to exchange of metal ions between polymer and nonpolysaccharide chelators. It is further believed that such exchange creates a dynamic pool of metal ions at the immunization site that is far more immunogenic than would be a static metal ion-chelate immunogen.

Many adjuvants are designed to cause persistence of the immunogenic compound in a depot at the site of injection, a process hereinafter termed “depotization”, in order to sustain presentation of antigen to the immune system over a longer period. The adjuvants used in the compositions of the present invention can usefully effect such depotization. The immunogenic composition of the present invention can also further include additional compounds that are believed to act further to “depotize” the antigen.

In one series of such embodiments, the compositions further comprise a crosslinked protein, wherein the protein has been crosslinked in the presence of the polymer chelator-metal complex, thereby enmeshing the immunogen. Without wishing to be bound by theory, it is believed that the crosslinked protein slows dissolution and dispersion of the metal ion-complexed polymer chelator, increasing the duration of presentation of the immunogen enmeshed therein to the immune system.

The protein can be any protein that is readily obtained and crosslinked.

However, if the protein is itself immunogenic, a principal advantage of the compositions and methods of the present invention, namely the substantial absence of antibodies that recognize a carrier component of the immunogen, will be lost.

Accordingly, the protein can usefully be nonxenogeneic to the host. By “nonxenogeneic” is intended a protein drawn from the same species as the host to be immunized. Where the host animal is a rabbit, for example, the protein can usefully be rabbit serum albumin (RSA). Nonxenogeneic proteins will prove less immunogenic than xenogeneic proteins.

The protein can be introduced into the immunogenic composition before or after addition of adjuvant, but is typically introduced before adjuvant addition.

The protein is then crosslinked using any protein crosslinker known in the art.

Common homobifunctional reagents that can be used include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available from Pierce, Rockford, Ill., USA). Common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (all available Pierce, Rockford, Ill., USA).

The protein can also conveniently be crosslinked using formaldehyde, glutaraldehyde, or glyoxal.

As mentioned, the protein can be introduced before or after addition of adjuvant. Where the protein is introduced prior to introduction of the adjuvant, the crosslinked protein/polymer chelator/metal ion composition is usefully dehydrated, such as by drying or lyophilization, before its dispersal in the adjuvant.

Although selection of nonxenogeneic proteins as the enmeshing agent reduces the potential for spurious antibody production, selection of a nonxenogeneic protein does not in itself solve the problems of protein solubility and cost. Furthermore, the protein is nonxenogeneic only with respect to a single species of host animal, precluding ready use of a single immunogenic composition for immunizing a plurality of species, as might be desired for production of both polyclonal and monoclonal antibodies.

The present inventors have discovered that certain gelatins that remain liquid at room temperature are remarkably well suited for inclusion in the immunogenic compositions of the present invention, being easily handled at room temperature, readily crosslinked, inexpensive, and poorly immunogenic in a variety of mammals.

Among such gelatins are gelatins that have been chemically modified to gel only at temperatures below room temperature, and gelatins that in their natural state gel only at temperatures below room temperature.

Among the latter are the gelatins of cold-water fish.

The ready commercial availability of fish gelatin renders fish gelatins particularly useful for inclusion in the immunogenic compositions of the present invention. Gelatins from cold-water fish are currently used in foods, particularly foods intended to meet dietary requirements of Jews and Muslims, and in glues. Gelatin from cold-water fish has also been described as useful in blocking nonspecific binding sites on nitrocellulose membranes used in immunoassays (Saravis, “Improved blocking of nonspecific antibody binding sites on nitrocellulose membranes,” Electrophoresis 5:54-55 (1984)).

Cold-water fish gelatin is commercially available as a pourable solution containing 45% solids in water (Norland HiPure Liquid Gelatin, Norland Products, Cranbury, N.J., USA). The gelatin remains liquid down to 8-10° C.

As described above, the fish gelatin can be added to the polymer chelator/metal complex before or after addition of adjuvant. Typically, the polymer chelator is contacted with metal ions for a time sufficient to permit binding of metal ions to the polymer chelator, and fish gelatin added thereafter. The gelatin is then crosslinked by addition of crosslinking agent, as above-described, usefully glutaraldehyde. Crosslinking is usefully conducted by slow addition of crosslinking agent, e.g. by dripwise addition. Thereafter, the immunogenic composition is dried, e.g. by lyophilization, and then dispersed in the adjuvant.

The resulting composition is a particulated, localized (depotized), dynamic metal ion immunogen that rapidly and cost-effectively elicits high titers of antibodies to the polymer chelator-metal ion chelate, without significant production of antibodies to the polymer chelator itself or to the gelatin meshwork. The immunogen is readily prepared and does not occasion appreciable morbidity in the immunized host.

The advantages of using crosslinked cold-water fish gelatin transcend its use with polymer chelator/metal ion complexes.

Accordingly, it is another aspect of the invention to provide immunogenic compositions that more generally comprise an immunogen enmeshed in a crosslinked gelatin. In typical embodiments, the gelatin is liquid at room temperature and crosslinked in the presence of the immunogen. The gelatin is typically a cold-water fish gelatin. We term such fish gelatin-enmeshed immunogens GEFILTEGEN™ immunogens.

The immunogen can be any immunogen currently used or contemplated by the art.

Where the immunogen is a small molecule incapable itself of eliciting a humoral immune response, i.e. a hapten, the small molecular weight molecule can be conjugated to a carrier, such as a protein different from the gelatin. Alternatively, the small molecular weight hapten can be conjugated directly to the gelatin, although the poor immunogenicity of the gelatin will often militate against its use directly as a carrier.

The gelatin can be crosslinked using any of the crosslinkers described above.

The immunogenic compositions of this aspect of the invention can further comprise an adjuvant, including any of the adjuvants above-described. Where an adjuvant is included, the protein can be crosslinked before or after, typically before, addition of adjuvant.

The immunogenic compositions of the present invention are capable of eliciting high titers of antibodies specific for the immunogen or for components thereof. It is, therefore, a further aspect of the present invention to provide methods for making an antibody reagent, the method comprising immunizing a nonhuman animal with the immunogenic compositions herein described, and isolating an antibody that binds specifically to the immunogenic composition. The serum concentration of antibodies specific for the immunogen elicited by the technique can be at least about 100 μg/ml, typically at least about 200 μg/ml, more typically at least about 500 μg/ml, and often up to at least about 1 mg/ml.

Protocols for immunizing nonhuman animals are well known in the art, and need not here be described in detail. See, e.g., Harlow et al. (eds.), Antibodies: A Laboratory Manual, Cold Spring Harbor Laboratory (1998) (ISBN: 0879693142); Coligan et al. (eds.), Current Protocols in Immunology, John Wiley & Sons, Inc. (2001) (ISBN: 0-471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907), the disclosures of which are incorporated herein by reference.

The choice of immunization protocol will depend in part upon the animal species chosen for immunization, which in turn will depend in part upon the choice as between production of polyclonal or monoclonal antibodies, the immunogenic compositions of the present invention being suitable for production of either monoclonal or polyclonal antibodies.

For applications that permit, polyclonal antibodies present significant advantages over monoclonal antibodies in terms of cost, time, and overall avidity.

Production of monoclonal antibodies of desired specificity can take as long as a year, and can be quite expensive. The cost of culture media alone often contributes significantly to the overall budget. Hybridomas can prove genetically unstable, obligating long term cryogenic storage of subclones as insurance against loss of secretion. For large scale production, hybridomas typically prove unsuitable, often obligating cloning of the Ig genes into a more suitable culture host, such as Chinese hamster ovary (CHO) cells.

Furthermore, although the monospecificity of mabs is prized for the resulting discriminatory power, such specificity will often come at the price of lowered total avidity, particularly for antigens that present a wide variety of antigenic epitopes.

Thus, polyclonal antibodies present certain advantages over monoclonal antibodies. Among these advantages are faster production, far lower cost, and often the ability to produce high titers of antibodies that collectively recognize a wide variety of analyte epitopes, thus providing a reagent having high avidity. Such high titer, high avidity reagents are particularly desired for diagnostic applications.

Where polyclonal antibodies are desired, the method of this aspect of the present invention comprises isolating antibodies from the serum of the immunized nonhuman animal.

As so isolated, the antibodies typically are contaminated with various other serum components, including proteins, lipids, carbohydrates, and inorganic molecules. Thus, the method of the present invention can optionally further comprise purification of the antibodies.

Purification can include, for example, dialysis or size exclusion chromatography to remove salts and other low molecular weight contaminants. Purification can include the selective adsorption of immunoglobulins in the protein fraction to reagents with high affinity for the Fc portion of immunoglobulins, such as Staph Protein A and Protein G. Purification can include affinity chromatography using the immunogen as the affinity moiety.

Purification can also include various types of negative selection, in which antibodies with affinity for other than the desired epitopes are absorbed and removed, thus creating a “monospecific polyclonal” reagent.

For example, where the immunogen is a polymer chelator-metal ion complex, absorption can be performed using the polymer chelator alone, effecting removal of antibodies that recognize polysaccharide epitopes that are independent of the metal ion component of the immunogen. Alternatively or in addition, absorption can be performed using the polymer chelator having a different metal ion bound thereto, removing antibodies that recognize the chelator alone and antibodies that recognize epitopes contributed by the alternative metal ion. By “contributed by” is intended epitopes of the metal ion itself or epitopes, typically conformational epitopes, created in the chelator by binding of a metal ion thereto.

Where, instead, the immunogen is a polypeptide, additional methods are available for preparing polyclonal antibodies of circumscribed specificity. See, e.g., Moshitch-Moshkovitz et al., J. Immunol. Methods 242(1-2):183-91 (2000); Brown-Augsburger et al., J. Pharm. Biomed Anal. 23(4):687-96 (2000); von Boxberg et al., Anal. Biochem. 219(1):32-6 (1994).

Purification is not obligatory, however. The immunogens of the present invention often are potent enough to produce titers of specific antibodies that permit a several-fold dilution to suffice to eliminate signal occasioned by the presence of antibodies having undesired specificities.

Where monoclonal antibodies are desired, the method of this aspect of the invention comprises a first step of isolating a cell that secretes an antibody that binds with specificity to the immunogenic composition.

Typically, the cell is a clonal cell line, such as a hybridoma, although methods that permit monoclonal antibody production without proceeding through hybridomas are known. See, e.g., U.S. Pat. No. 5,627,052, the disclosure of which is incorporated herein by reference in its entirety.

Methods of producing and screening hybridomas are well known in the art, and are amply well described in Harlow et al. (eds.), Antibodies: A Laboratory Manual, Cold Spring Harbor Laboratory (1998) (ISBN: 0879693142); Coligan et al. (eds.), Current Protocols in Immunology, John Wiley & Sons, Inc. (2001) (ISBN: 0-471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907), the disclosures of which are incorporated herein by reference.

Where the immunogen is a polymer chelator-metal ion complex, screening can be performed using the original polymer chelator-metal ion immunogen, with counterscreening performed with the polymer chelator alone and/or with the polymer chelator complexed to a different metal ion.

Screening will identify individual clones that secrete antibodies of desired specificity. Thereafter, the antibody reagent can be isolated directly from the cell culture media.

Alternatively, the antibody reagent can be isolated after one or more intermediary steps, which steps effect recombinant expression of the encoding genes in a new host cell. Recombinant expression of antibodies in host cells is particularly useful when fragments or derivatives of the antibodies of the present invention are desired.

Host cells for recombinant antibody production—either whole antibodies, antibody fragments, or antibody derivatives—can be prokaryotic or eukaryotic.

Prokaryotic hosts are particularly useful for producing phage displayed antibodies.

The technology of phage-displayed antibodies, in which antibody variable region fragments are fused, for example, to the gene III protein (pIII) or gene VIII protein (pVIII) for display on the surface of filamentous phage, such as M13, is by now well-established, Sidhu, Curr. Opin. Biotechnol. 11(6):610-6 (2000); Griffiths et al., Curr. Opin. Biotechnol. 9(1):102-8 (1998); Hoogenboom et al., Immunotechnology, 4 (1):1-20 (1998); Rader et al., Current Opinion in Biotechnology 8:503-508 (1997); Aujame et al., Human Antibodies 8:155-168 (1997); Hoogenboom, Trends in Biotechnol. 15:62-70 (1997); de Kruif et al., 17:453-455 (1996); Barbas et al., Trends in Biotechnol. 14:230-234 (1996); Winter et al., Ann. Rev. Immunol. 433-455 (1994), and techniques and protocols required to generate, propagate, screen (pan), and use the antibody fragments from such libraries have recently been compiled, Barbas et al., Phage Display: A Laboratory Manual, Cold Spring Harbor Laboratory Press (2001) (ISBN 0-87969-546-3); Kay et al. (eds.), Phage Display of Peptides and Proteins: A Laboratory Manual, Academic Press, Inc. (1996); Abelson et al. (eds.), Combinatorial Chemistry, Methods in Enzymology vol. 267, Academic Press (May 1996), the disclosures of which are incorporated herein by reference in their entireties.

Typically, phage-displayed antibody fragments are scFv fragments or Fab fragments; when desired, full length antibodies can be produced by cloning the variable regions from the displaying phage into a complete antibody and expressing the full length antibody in a further prokaryotic or a eukaryotic host cell.

Eukaryotic cells are also useful for expression of the antibodies, antibody fragments, and antibody derivatives of the present invention.

For example, antibody fragments of the present invention can be produced in Pichia pastoris, Takahashi et al., Biosci. Biotechnol. Biochem. 64(10):2138-44 (2000); Freyre et al., J. Biotechnol. 76(2-3):157-63 (2000); Fischer et al., Biotechnol. Appl. Biochem. 30 (Pt 2):117-20 (1999); Pennell et al., Res. Immunol. 149(6):599-603 (1998); Eldin et al., J. Immunol. Methods. 201(1):67-75 (1997); and in Saccharomyces cerevisiae, Frenken et al., Res. Immunol. 149(6):589-99 (1998); Shusta et al., Nature Biotechnol. 16(8):773-7 (1998), the disclosures of which are incorporated herein by reference in their entireties.

Antibodies, including antibody fragments and derivatives, of the present invention can also be produced in insect cells, Li et al., Protein Expr. Purif. 21(1):121-8 (2001); Ailor et al., Biotechnol. Bioeng. 58(2-3):196-203 (1998); Hsu et al., Biotechnol. Prog. 13(1):96-104 (1997); Edelman et al., Immunology 91(1):13-9 (1997); and Nesbit et al., J. Immunol. Methods. 151(1-2):201-8 (1992), the disclosures of which are incorporated herein by reference in their entireties.

Antibodies and fragments and derivatives thereof of the present invention can also be produced in plant cells, Giddings et al., Nature Biotechnol. 18(11):1151-5 (2000); Gavilondo et al., Biotechniques 29(1):128-38 (2000); Fischer et al., J. Biol. Regul. Homeost. Agents 14(2):83-92 (2000); Fischer et al., Biotechnol. Appl. Biochem. 30 (Pt 2):113-6 (1999); Fischer et al., Biol. Chem. 380(7-8):825-39 (1999); Russell, Curr. Top. Microbiol. Immunol. 240:119-38 (1999); and Ma et al., Plant Physiol. 109(2):341-6 (1995), the disclosures of which are incorporated herein by reference in their entireties.

Mammalian cells useful for recombinant expression of antibodies, antibody fragments, and antibody derivatives of the present invention include CHO cells, COS cells, 293 cells, and myeloma cells.

Verma et al., J. Immunol. Methods 216(1-2): 165-81 (1998), review and compare bacterial, yeast, insect and mammalian expression systems for expression of antibodies.

Antibodies of the present invention can also be prepared by cell free translation, as further described in Merk et al., J. Biochem. (Tokyo). 125(2):328-33 (1999) and Ryabova et al., Nature Biotechnol. 15(1):79-84 (1997), and in the milk of transgenic animals, as further described in Pollock et al., J. Immunol. Methods 231(1-2):147-57 (1999), the disclosures of which are incorporated herein by reference in their entireties.

As noted, recombinant expression is particularly useful when fragments and derivatives of the antibodies of the present invention are desired.

Among such useful fragments are Fab, Fab′, Fv, F(ab)′2, and single chain Fv (scFv) fragments. Other useful fragments are described in Hudson, Curr. Opin. Biotechnol. 9(4):395-402 (1998).

Among useful derivatives are chimeric, primatized, and humanized antibodies; such derivatives are less immunogenic in human beings, and thus more suitable for in vivo administration, than are unmodified antibodies from non-human mammalian species.

Chimeric antibodies typically include heavy and/or light chain variable regions (including both CDR and framework residues) of immunoglobulins of one species, typically mouse, fused to constant regions of another species, typically human. See, e.g., U.S. Pat. No. 5,807,715; Morrison et al., Proc. Natl. Acad. Sci USA. 81(21):6851-5 (1984); Sharon et al., Nature 309(5966):364-7 (1984); Takeda et al., Nature 314(6010):452-4 (1985), the disclosures of which are incorporated herein by reference in their entireties.

Primatized and humanized antibodies typically include heavy and/or light chain CDRs from a murine antibody grafted into a non-human primate or human antibody V region framework, usually further comprising a human constant region, Riechmann et al., Nature 332(6162):323-7 (1988); Co et al., Nature 351(6326):501-2 (1991); U.S. Pat. Nos. 6,054,297; 5,821,337; 5,770,196; 5,766,886; 5,821,123; 5,869,619; 6,180,377; 6,013,256; 5,693,761; and 6,180,370, the disclosures of which are incorporated herein by reference in their entireties.

Other useful antibody derivatives of the invention include heteromeric antibody complexes and antibody fusions, such as diabodies (bispecific antibodies), single-chain diabodies, and intrabodies.

After secretion, whether by a hybridoma or a recombinantly engineered expression host, the monoclonal antibody can optionally be purified.

Monoclonal antibodies typically will not require affinity-based purification to remove antibodies having unrelated specificities. Nonetheless, as is well known in the art, purification of the antibody from other proteins present in the culture medium will often be desired, and can be effected by absorption using Fc-specific reagents, such as Protein A or Protein G, or by affinity purification using the original immunogen.

Where the immunogen includes a polymer-chelated metal ion, the antibodies—whether polyclonal or monoclonal—will typically bind to the metal ion as complexed to the polymer chelator used for immunization. Where a plurality of chelators are included in the immunogen, including, e.g., both a polymer chelator and a nonpolysaccharide chelator, the resulting antibodies may primarily, but not exclusively, recognize the metal ion as complexed to a variety of different chelators. Antibodies against free metal ions may also be generated by the immunization protocol.

The antibody reagents of the present invention—whether polyclonal or monoclonal, native protein or fragment or derivative thereof—exhibit specific binding to the original immunogen, discriminating over adventitious binding interactions by at least two-fold, more typically by at least 2-fold, more typically by at least 5-fold, typically by more than 10-fold, 25-fold, 50-fold, 75-fold, and often by more than 100-fold, and on occasion by more than 500-fold or 1000-fold.

Typically, the affinity or avidity of the antibodies (or antibody multimers, as in the case of an IgM pentamer) of the present invention for the immunogen will be at least about 1×10⁻⁶ molar (M), typically at least about 5×10⁻⁷ M, usefully at least about 1×10⁻⁷ M, with affinities and avidities of at least 1×10⁻⁸ M, 5×10⁻⁹ M, and 1×10⁻¹⁰ M proving especially useful.

The antibodies of the present invention, including fragments and derivatives thereof, can usefully be labeled. It is, therefore, another aspect of the present invention to provide labeled antibodies that bind specifically to the immunogen, or the binding of which can be competitively inhibited by the immunogen.

The choice of label depends, in part, upon the desired use.

For example, when the antibodies of the present invention are used for immunohistochemical staining of tissue samples, the label can usefully be an enzyme that catalyzes production and local deposition of a detectable product.

Enzymes typically conjugated to antibodies to permit their immunohistochemical visualization are well known, and include alkaline phosphatase, β-galactosidase, glucose oxidase, horseradish peroxidase (HRP), and urease. Typical substrates for production and deposition of visually detectable products include o-nitrophenyl-beta-D-galactopyranoside (ONPG); o-phenylenediamine dihydrochloride (OPD); p-nitrophenyl phosphate (PNPP); p-nitrophenyl-beta-D-galactopryanoside (PNPG); 3′,3′-diaminobenzidine (DAB); 3-amino-9-ethylcarbazole (AEC); 4-chloro-1-naphthol (CN); 5-bromo-4-chloro-3-indolyl-phosphate (BCIP); ABTS®; BluoGal; iodonitrotetrazolium (INT); nitroblue tetrazolium chloride (NBT); phenazine methosulfate (PMS); phenolphthalein monophosphate (PMP); tetramethyl benzidine (TMB); tetranitroblue tetrazolium (TNBT); X-Gal; X-Gluc; and X-Glucoside.

Other substrates can be used to produce products for local deposition that are luminescent. For example, in the presence of hydrogen peroxide (H₂O₂), horseradish peroxidase (HRP) can catalyze the oxidation of cyclic diacylhydrazides, such as luminol. Immediately following the oxidation, the luminol is in an excited state (intermediate reaction product), which decays to the ground state by emitting light. Strong enhancement of the light emission is produced by enhancers, such as phenolic compounds. Advantages include high sensitivity, high resolution, and rapid detection without radioactivity. See, e.g., Thorpe et al., Methods Enzymol. 133:331-53 (1986); Kricka et al., J. Immunoassay 17(1):67-83 (1996); and Lundqvist et al., J. Biolumin. Chemilumin. 10(6):353-9 (1995), the disclosures of which are incorporated herein by reference in their entireties. Kits for such enhanced chemiluminescent detection (ECL) are available commercially.

The antibodies can also be labeled using colloidal gold.

As another example, when the antibodies of the present invention are used, e.g., for flow cytometric detection, for scanning laser cytometric detection, or for fluorescent immunoassay, they can usefully be labeled with fluorophores.

There are a wide variety of fluorophore labels that can usefully be attached to the antibodies of the present invention.

For flow cytometric applications, both for extracellular detection and for intracellular detection, common useful fluorophores can be fluorescein isothiocyanate (FITC), allophycocyanin (APC), R-phycoerythrin (PE), peridinin chlorophyll protein (PerCP), Texas Red, Cy3, Cy5, fluorescence resonance energy tandem fluorophores such as PerCP-Cy5.5, PE-Cy5, PE-Cy5.5, PE-Cy7, PE-Texas Red, and APC-Cy7.

Other fluorophores include, inter alia, Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3, Cy3.5, Cy5, Cy5.5, Cy7, all of which are also useful for fluorescently labeling the antibodies of the present invention.

For secondary detection using labeled avidin, streptavidin, captavidin or neutravidin, the antibodies of the present invention can usefully be labeled with biotin.

When the antibodies of the present invention are used, e.g., for Western blotting applications, they can usefully be labeled with radioisotopes, such as ³³P, ³²P, ³⁵S, ³H, and ¹²⁵I.

As yet another example, when the antibodies of the present invention are used for radioimmunotherapy, the label can usefully be ²²⁸Th, ²²⁷Ac, ²²⁵Ac, ²²³Ra, ²¹³Bi, ²¹²Pb, ²¹²Bi, ²¹¹At, ²⁰³Pb, ¹⁹⁴OS, ¹⁸⁸Re, ¹⁸⁶Re, ¹⁵³Sm, ¹⁴⁹Tb, ¹³¹I, ¹²⁵I, ¹¹¹In, ¹⁰⁵Rh, ⁹⁹mTc, ⁹⁷Ru, ⁹⁰Y, ⁹⁰Sr, ⁸⁸Y, ⁷²Se, ⁶⁷CU, or ⁴⁷SC.

As another example, when the antibodies of the present invention are to be used for in vivo diagnostic use, they can be rendered detectable by conjugation to MRI contrast agents, such as gadolinium diethylenetriaminepentaacetic acid (DTPA), Lauffer et al., Radiology 207(2):529-38 (1998), or by radioisotopic labeling

As would be understood, use of the labels described above is not restricted to the application as for which they are above-mentioned.

The antibodies of the present invention, including fragments and derivatives thereof, can also be conjugated to toxins, in order to target the toxin's ablative action to cells that display or otherwise contain the immunogen.

Commonly, the antibody in such immunotoxins is conjugated to Pseudomonas exotoxin A, diphtheria toxin, shiga toxin A, anthrax toxin lethal factor, or ricin. See Hall (ed.), Immunotoxin Methods and Protocols (Methods in Molecular Biology, Vol 166), Humana Press (2000) (ISBN:0896037754); and Frankel et al. (eds.), Clinical Applications of Immunotoxins, Springer-Verlag New York, Incorporated (1998) (ISBN:3540640975), the disclosures of which are incorporated herein by reference in their entireties, for review.

The antibodies of the present invention can usefully be attached to a substrate and it is, therefore, another aspect of the invention to provide antibodies with specificity for the immunogens of the present invention, as attached to a substrate.

Substrates can be porous or nonporous, planar or nonplanar.

For example, the antibodies of the present invention can usefully be conjugated to filtration media, such as NHS-activated Sepharose or CNBr-activated Sepharose for purposes of immunoaffinity chromatography.

For example, the antibodies of the present invention can usefully be attached to paramagnetic microspheres, typically by biotin-streptavidin interaction, which microsphere can then be used for isolation of cells that display the immunogen of the present invention. As another example, the antibodies of the present invention can usefully be attached to the surface of a microtiter plate for ELISA.

As noted above, the antibodies of the present invention can be produced in prokaryotic and eukaryotic cells. It is, therefore, another aspect of the present invention to provide cells that express the antibodies of the present invention, including hybridoma cells, B cells, plasma cells, and host cells recombinantly modified to express the antibodies of the present invention.

Applications of Antibodies Having Specificity for Metal Ions and Metal Ion Chelates

The compositions and methods of the present invention are capable of eliciting high titers of antibodies to low molecular weight analytes. Many such antibodies, such as those with specificity for metal ions and chelates thereof, are useful in clinical monitoring and diagnosis.

For such diagnosis or monitoring applications, the antibodies can usefully be incorporated into devices that extract the analyte transdermally, obviating the invasive obtention of fluids from the patient.

Devices for rapid transdermal extraction of analytes are described in copending and commonly owned U.S. patent application Ser. No. 09/339,147, filed Jun. 24, 1999, the disclosure of which is incorporated herein by reference in its entirety.

The devices comprise an absorbant or a hydrogel, the absorbant or hydrogel having within it a pyrrolidone of the following formula in aqueous admixture,

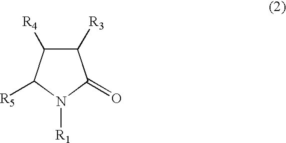

wherein R₁=H, CH₃—, HO—CH₂—CH₂—, CH₃—CH₂—, or CH₃—(CH₂)n-, wherein n=1 to 11; R₃=H, —OH, CH₃—, or CH₃—(CH₂)n-, wherein n=1 to 11; R4=H, or methyloxycarbonyl, and R₅=H, CH₃—, CH₃—CH₂—, or HO—CH₂—CH₂. The pyrrolidone is present in the mixture at an aqueous concentration of at least about 25% (v/v).

The pyrrolidone can usefully be selected from the group consisting of N-methyl 2-pyrrolidone, 2-pyrrolidone, 1-methyl-2-pyrrolidone, 1-ethyl-2-pyrrolidone, 1-hexyl-2-pyrrolidone, 1-lauryl-2-pyrrolidone, N-(2-hydroxyethyl)-2-pyrrolidone, 1,5-dimethyl-2-pyrrolidone, 5-methyl-2-pyrrolidone, 1-hexyl-4-methyloxycarbonyl-2-pyrrolidone, 1-lauryl-4-methyloxycarbonyl-2-pyrrolidone, N-cyclohexyl-2-pyrrolidone, N-dodecyl-2-pyrrolidone, and 1-butyl-3-dodecyl-2-pyrrolidone.

In certain preferred embodiments, the pyrrolidone is N-methyl 2-pyrrolidone (NMP) or 2-pyrrolidone, often NMP.

The pyrollidone can be admixed with water, with saline, or with aqueous buffer, often at an aqueous concentration (v/v pyrrolidone:aqueous phase) of no more than about 75%, often about 50%.

In these transdermal extraction devices, the aqueous pyrrolidone mixture is included within a hydrogel or absorbant.

The hydrogel can usefully comprise a polymer selected from the group consisting of poloxamers, polyvinyl alcohol, polyhydroxymethacrylate, polyhydroxyethyl methacrylate, propylene glycol and acrylamide. Among poloxamers, poloxamer 407 proves particularly useful. Absorbants can include, e.g., a cellulosic pad or gauze patch.

As disclosed in copending and commonly owned U.S. patent application Ser. No. 09/339,147, filed Jun. 24, 1999, N-methyl 2-pyrrolidone (NMP) and related structural analogues, known in the art to enhance skin permeability, have surprisingly been found to be far more potent in facilitating transdermal diffusion when formulated in aqueous, rather than organic (or lipophilic) admixture; additionally, the potency of these aqueous formulations, that is, their ability to enhance outward diffusion of analytes present in interstitial fluids, is shown therein to be strikingly dependent on the enhancing agent's aqueous concentration.

Properly formulated, therefore, the aqueous pyrrolidones sufficiently enhance the outward diffusion of analytes as to permit the simple, noninvasive, transdermal detection of analytes—without the adjunctive use of physical perturbants, such as ultrasound or electroporation—in as few as 24 hours, and in some cases in as few as 30-60 minutes.

The antibodies of the present invention are usefully incorporated into such transdermal extraction devices in operative association with detection means, thus creating a transdermal detection device. The detection means can be qualitative or quantitative.

In the alternative, the analyte extracted transdermally into the patch can be detected by detection means discrete from the patch itself. The antibodies of the present invention are usefully incorporated into such detection means.

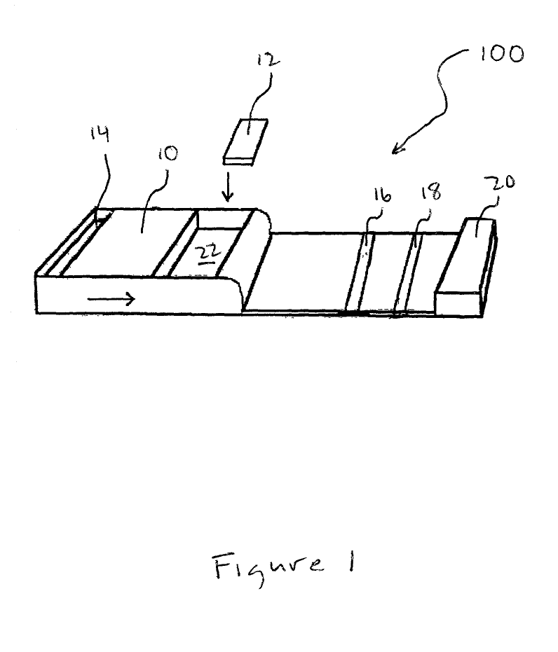

For example, FIG. 1 provides a schematic of a lateral flow device 100 for the detection and measurement of lead that has been extracted transdermally into a hydrogel.

As shown, rabbit anti-lead antibodies, labeled with colloidal gold or other detectable marker, are diffusibly included in support 10 at a first position in device 100. Hydrogel patch 12 is placed at a second position 22 in device 100 as indicated. An aqueous solution placed in reservoir 14 flows laterally through support 10 to promote diffusion and mixing of solutes from support 10 with lead ions eluted from hydrogel patch 12. The solution, which carries the labeled antibody, tightly bound to any lead ions eluted from hydrogel patch 12, flows past a first detection zone 16 that contains immobilized rabbit anti-lead antibodies or lead-chelating beads. The solution then flows past a second detection zone 18 that contains immobilized antibodies specific for the rabbit antibodies, or another agent, such as Protein A, that will bind specifically to antibodies. Flow of the aqueous solution through device 100 is facilitated by absorbant pad 20.

The presence of lead ions in hydrogel patch 12 is detected by the specific binding of the labeled anti-lead antibodies at first detection zone 16. The amounts of labeled anti-lead antibodies in device 100 can be adjusted as desired so that a specific amount of lead ions in hydrogel patch 12 will result in the binding of all of the labeled anti-lead antibodies at first detection zone 16. Smaller amounts of lead ions in hydrogel patch 12 will result in partial binding of the labeled anti-lead antibodies at the first detection zone 16, with the excess labeled anti-lead antibodies being bound at second detection zone 18. If no lead ions are present in hydrogel patch 12, all of the labeled anti-lead antibodies will flow past first detection zone 16 and will be bound at second detection zone 18. The second detection zone 18 thus serves as a control to confirm that the labeled anti-lead antibodies have flowed through device 100.

In one example of this detection scheme, chelating beads prepared as above-described can be placed within the transdermal extraction patch to bind to lead ions as they are extracted transdermally into the hydrogel. Anti-lead antibodies prepared as above-described can then be used in lateral flow device 100 to detect the chelated lead ions as shown in FIG. 1.

The present invention will be further understood by reference to the following non-limiting examples.

### EXAMPLE 1

### Preparation of Anti-Lead Antibodies Using Alginate Beads as a Naturally Occurring Metal-Binding Nonimmunogenic Polymer

This Example demonstrates that alginate can be used directly to complex lead ions, and that the alginate-lead ion complex proves remarkably effective as an immunogen for preparing high titers of anti-lead polyclonal antibodies.

Methods

Preparation of Metal Ion-Chelating Beads

Alginate-containing agarose beads are prepared essentially as described in U.S. Pat. No. 6,248,268 and WO 00/29466.

Briefly, Gracilaria-derived agarose, type D-2 (Hispanagar SA, Spain) is slowly added to cold, distilled water to a final concentration of 1% (w/v). Low viscosity alginate (ISP Alginates, Inc., San Diego, Calif. or TIC Gums, Inc., Belcamp, Md.) is added. The aqueous mixture is heated to boiling until all components are thoroughly dissolved, and then held at 85° C. The molten sol is then sprayed at a temperature of 70° C. into ambient air, and gelled particles collected. The agarose-alginate beads are then crosslinked using divinylsulfone.

The beads have diameter of about 50-250 μm, and are estimated to have a binding capacity for divalent cations of at least about 24-30 μmoles per ml of drained beads.

Preparation of Lead Ion Chelate

A lead ion chelate is formulated by combining the alginate-agarose beads with a stoichiometric quantity of lead ions in the form of lead acetate, as follows:

Two (2) grams of lead acetate are added to a chelating mixture totaling a final weight of approximately 23 grams. This is formulated into an aqueous liquid gel suspension. The gel is lyophilized to a dry cake. The cake is pulverized to a fine powder so that it could pass through a 21 gauge needle. The final chelate contains approximately 8.3% lead acetate per unit mass of the polysaccharide-lead chelate. Thus, there is about 100 μg lead acetate in 1.2 mg of chelate.

Immunization

Approximately 100 micrograms of chelated lead acetate immunogen is injected per rabbit per injection according to the following schedule:

The chelate immunogen (4.8 mg) is suspended into 1 ml of phosphate buffered saline (“PBS”). One (1) ml of Complete Freund's Adjuvant is added, and this is mixed well. Of the mixture, 0.5 ml is injected into each of four (4) rabbits in the muscle of the hind leg. Fourteen days later, another 4.8 mg of the chelated immunogen is added to 1 ml of PBS and 1 ml of aqueous aluminum magnesium hydroxide (MAALOX™). This is mixed well and 0.5 ml of the mixture is injected intramuscularly (IM) into each of the four rabbits. Seven days later, the rabbits are trial bled and tested for circulating antibodies to chelated lead.

This schedule is repeated approximately every two weeks over a 151 day period using the aqueous aluminum magnesium hydroxide mixture until a useful titer of anti-lead antibodies is obtained.

Titering

Antibody titers are measured using an ELISA methodology, as follows:

One hundred microliters (100 μL) of immunogen (the solid phase lead ion-chelating bead preparation, as above) at a concentration of 9 μg/ml is used to coat each well of a 96 well ELISA plate. The wells are washed with phosphate buffered saline (PBS). Nonspecific binding sites are blocked with 1% bovine serum albumin (“BSA”). The test bleeds of the rabbits are reacted for 1 hour with the washed chelating bead preparation. The beads are then washed three times with PBS containing 1% BSA and then reacted with goat anti-rabbit IgG (H+L) antiserum conjugated with horseradish peroxidase. Binding of the second phase antibody is assayed in the presence of ABTS (2,2′-azino-bis(3-ethylbenzthiazoline-6-sulfonic acid) (detecting dye). The solutions are then read at 450 nm.

Results

No adverse reactions to the injection of the immunogen are observed at any time in any of the rabbits. Titers calculated using the ELISA assay are set forth in Table 1.

TABLE 1

| Day | Rabbit # | Antigen | Antibody Titer |
|---|---|---|---|
| 0 | 1 | Chelated lead | 0 |
| 0 | 2 | Chelated lead | 0 |
| 0 | 3 | Chelated lead | 0 |
| 0 | 4 | Chelated lead | 0 |
| 21 | 1 | Chelated lead | 1:1000 |
| 21 | 2 | Chelated lead | 1:1000 |
| 21 | 3 | Chelated lead | 1:5000 |
| 21 | 4 | Chelated lead | 1:1000 |
| 40 | i | Chelated lead | 1:50,000 |
| 40 | 2 | Chelated lead | 1:50,000 |
| 40 | 3 | Chelated lead | 1:70,000 |
| 40 | 4 | Chelated lead | 1:50,000 |
| 76 | 1 | Chelated lead | 1:100,000 |
| 76 | 2 | Chelated lead | 1:500,000 |
| 76 | 3 | Chelated lead | 1:1,000,000 |
| 76 | 4 | Chelated lead | 1:100,000 |
| 109 | 1 | Chelated lead | 1:500,000 |
| 109 | 2 | Chelated lead | 1:1,000,000 |
| 109 | 3 | Chelated lead | 1:1,000,000 |
| 109 | 4 | Chelated lead | 1:500,000 |
| 133 | 1 | Chelated lead | 1:300,000 |
| 133 | 2 | Chelated lead | 1:300,000 |
| 133 | 3 | Chelated lead | 1:500,000 |
| 133 | 4 | Chelated lead | 1:500,000 |
| 151 | 1 | Chelated lead | 1:1,000,000 |
| 151 | 2 | Chelated lead | 1:1,000,000 |
| 151 | 3 | Chelated lead | 1:1,000,000 |
| 151 | 4 | Chelated lead | 1:1,000,000 |

The results show that the alginate-lead ion chelate results in the production of high titers of antibodies that recognize the immunogen, i.e., lead ions directly chelated by alginate. Titers are calculated using a colorimetric ELISA, without enhancement, further indicating that very good polyclonal antibodies are being generated.

### EXAMPLE 2

### Improved Lead Immunogen

This Example illustrates the preparation of an improved immunogen in which the alginate-lead ion chelate is enmeshed within a crosslinked protein, with the further inclusion of a nonpolysaccharide chelator, EDTA.

The improved lead ion immunogen is prepared as follows:

1. Wash alginate-containing agarose beads prepared as set forth in Example 1 with PBS, 0.15 M, pH 7.2

2. Add beads to 7 mL acetate buffer, 0.2 M, pH 5

3. Add 2 gms Pb acetate and mix

4. Add 4 gms tetrasodium EDTA and mix

5. Add and mix 2 gms of lyophilized rabbit serum albumin (or other traditional carrier proteins)

6. Dropwise add 3 mL 2.5% glutaraldehyde

7. Let stand 3 hours

8. Macerate and add a dilution of 10% rabbit serum albumin (or other traditional blocking proteins)

9. Wash with distilled water

10. Make a suspension in distilled water and lyophilize to dryness using a filter trap

11. In a hood grind to fineness

12. Weigh, add to adjuvant, inject

The acetate buffer is prepared as a 2M stock as follows: sodium acetate (NaC₂H₃O₂.3H₂O), 24 gms; glacial acetic acid, 1.32 mL; distilled water to 100 mL. The glutaraldehyde solution is made from 25% stock and is added dropwise to the gently stirred albumin solution. The reaction mixture is then allowed to stand for three hours at room temperature without stirring. A gel usually appears 10 to 30 min later.

The serum albumin proteins that are included in the improved immunogen are nonxenogeneic: i.e., they are derived from the same species (rabbit) into which the immunogen will be inoculated. Typically, such nonxenogeneic albumins do not elicit high titer antibodies specific for any denatured albumin in the immunogen.

Any such anti-albumin antibodies that are produced are removed by absorption, for example by absorption to solid phase rabbit albumin lacking Pb acetate. In the alternative or in addition, the anti-lead antibodies are purified from anti-albumin (and other nonspecific) antibodies by affinity selection, for example by passing the antisera through a column of lead ion-alginate bead complex, then eluting the specific anti-lead antibodies for test purposes.

### EXAMPLE 3

### Improved Metal Immunogens Using Fish Gelatin

This Example illustrates the preparation of improved immunogens in which the alginate-lead chelate is enmeshed within crosslinked fish gelatin, further including EDTA.

Lead Immunogen Preparation

1. Wash metal-chelating alginate beads prepared as in Example 1 with PBS, 0.15 M, pH 7.2

2. Add alginate beads to 7 mL acetate buffer, 0.2 M, pH 5

3. Add 2 gms Pb acetate and mix

4. Add 4 gms tetrasodium EDTA and mix

5. Add and mix 2 gms (2 mL) liquid fish gelatin (Norland Products Inc., 2540 Route 130, Cranbury, N.J.)

6. Dropwise add 3 mL 2.5% glutaraldehyde

7. Let stand 3 hours

8. Macerate a nd add 10% (w/v) fish gelatin

9. Wash with distilled water

10. Make a suspension in distilled water and lyophilize to dryness using a filter trap

11. In a hood grind to fineness

12. Weigh, add to adjuvant, inject

The acetate buffer is prepared as a 2M stock as follows: sodium acetate (NaC₂H₃O₂.3H₂O), 24 gms; glacial acetic acid, 1.32 mL; distilled water to 100 mL. The glutaraldehyde solution is made from 25% stock and is added dropwise to the gently stirred albumin solution. The reaction mixture then is allowed to stand for three hours at room temperature without stirring. The immunogen typically gels 10 to 30 min later.

Mercury Immunogen Preparation

1. Wash metal-chelating alginate beads prepared as in Example 1 with PBS, 0.15 M, pH 7.2

2. Centrifuge at 800×g for 1 min, then add 2 mL of the packed, washed beads to 7 mL acetate buffer, 0.2 M, pH 5

3. Add 2 gms Hg acetate and mix

4. Rapidly add 4 gms tetrasodium EDTA and mix

5. Rapidly add and mix 4 mL liquid fish gelatin (Norland Products Inc., 2540 Route 130, Cranbury, N.J.)

6. Dropwise add 5 mL 2.5% glutaraldehyde

7. Let stand 3 hours

8. Macerate and add 10% (w/v) fish gelatin

9. Wash with distilled water

10. Make a suspension in distilled water and lyophilize to dryness using a filter trap

11. In a hood, grind to fineness

12. Weigh, add to adjuvant, inject

The glutaraldehyde solution is made from 25% stock and is added dropwise to the gently stirred mercury gelatin solution. The reaction mixture is then allowed to stand for three hours at room temperature without stirring. A gel usually appears within a few minutes.

The acetate buffer is prepared as a 2M stock as follows: sodium acetate (NaC₂H₃O₂.3H₂O), 24 gms; glacial acetic acid, 1.32 mL; distilled water to 100 mL.

Immunization with Mercury Chelate

Rabbits are injected on DAY 0, DAY 21 and DAY 31 with the mercury-alginate-fish gelatin-EDTA complex prepared as described above. Blood samples are taken on day 42 and the serum is assayed via ELISA using mercury chelate-coated plates. In order to determine the specificity of the resulting antibodies for mercury chelate over chelate alone, the sera are also evaluated via ELISA using plates coated with lead chelate prepared as described above.

The following table summarizes the observed reactions in the presence of mercury chelate and lead chelate:

TABLE 2

| | | Antibody Titer via ELISA | |
|---|---|---|---|
| Mercury Chelate ID | Animal # | Mercury Chelate | Lead Chelate |
| TDTO1-01A | 1 | 1:100,000 | 1:10,000 |
| | 2 | 1:100,000 | 1:10,000 |
| | 3 | 1:50,000 | 1:1,000 |
| | 4 | 1:100,000 | 1:10,000 |
| TDTO1-01B | 1 | 1:100,000 | 1:10,000 |
| | 2 | 1:100,000 | 1:10,000 |
| | 3 | 1:100,000 | 1:50,000 |
| | 4 | 1:50,000 | 1:10,000 |
| TDTO1-01C | 1 | 1:10,000 | 1:100,000 |
| | 2 | 1:50,000 | 1:10,000 |
| | 3 | 1:1,000 | 1:50,000 |
| | 4 | 1:100,000 | 1:100,000 |
| TDTO1-01D | 1 | 1:100,000 | 1:100,000 |
| | 2 | 1:50,000 | 1:100,000 |
| | 3 | 1:50,000 | 1:100,000 |
| | 4 | 1:100,000 | 1:100,000 |

Mercury chelate preparations TDTO1-01A and TDTO1-01B produce high titers of antibodies that show greater specificity for mercury chelate than for lead chelate.

Immunization with lead chelate

Rabbits are injected with lead chelate prepared as described above.

Serum from one rabbit, having an ELISA titer vs. lead chelate of 1:100,000, is used to determine the specificity for chelate lead as compared to specificity for the polymer chelator lacking lead.

One aliquot of the serum is incubated overnight (25° C.) on a rotator at a 1:1 (vol/vol) concentration with the immunogen, lead chelate gel beads. A second aliquot of the serum is diluted 1:1 with PBS (i.e., without absorbant) and incubated overnight at 25° C. on a rotator.

An ELISA assay is run with these two aliquots, using untreated serum as a further positive control. The ELISA is performed using both lead chelate-treated plates and mercury chelate-treated plates to determine the specificity of the antisera for chelated lead over chelated mercury and the polymer chelator alone. The following table summarizes the observed reactions in the presence of lead chelate and mercury chelate.

TABLE 3

| | Antibody Titer via ELISA | | |
|---|---|---|---|
| Chelate | Untreated Serum | Serum/PBS | Serum/Beads |
| Lead | 1:50,000 | 1:50,000 | — |
| Mercury | 1:1,000 | 1:5,000 | 1:1000 |

The results show that the antisera produced against the lead chelate is selective for the lead-charged complex over the lead-free polymer chelator.

### EXAMPLE 4

### Fish Gelatin Immunogens

This Example demonstrates the preparation of immunogens to low molecular weight molecules other than metal ions, including proteins of low immunogenicity separated by agarose electrophoresis, using crosslinked fish gelatin.

1. Isolate specific small molecular weight peptide or protein molecules by electrophoresis in an agarose gel. Cut the desired molecules from an agarose gel, macerate, and dilute with sodium bicarbonate buffer (0.1M, pH 8.3) containing 0.5M NaCl at a 1:5 dilution. (The presence of agarose electrophoresis gel does not inhibit antibody formation).

2. Place with cyanogen bromide (CNBr)-activated Sepharose 4B (about 5-10 mg protein per mL swollen gel).

3. Incubate overnight at 4° C. in an end-over-end mixer.

4. Do not wash the beads. Centrifuge the beads and add to 7 mL acetate buffer, 0.2 M, pH 5.

5. Add 2 gms fish gelatin and mix.

6. Dropwise add 3 mL 2.5% glutaraldehyde

7. Let stand 3 hours

8. Macerate and add 10% fish gelatin

9. Wash with distilled water

10. Make a suspension in distilled water and lyophilize to dryness using a filter trap

11. In a hood, grind to fineness

12. Weigh, add to adjuvant. A suspension of beads containing approximately 2 mg of peptide or protein is mixed with an equal volume of Complete Freunds' Adjuvant.

13. Inject 0.5 mL I.M. in the hind quarters or rabbits.

Two weeks later, mix an aliquot of the bead suspension with an equal volume of aqueous aluminum magnesium hydroxide gel (e.g., Amphogel, Maalox) and inject 0.5 mL I.M. in the hind quarters of the previously injected rabbits.

Trial bleed seven days later and inject another aliquot of the aluminum magnesium hydroxide gel bead suspension as described above.

Coupling of antigen to the Sepharose beads serves to protect labile proteins from bacterial and enzymatic damage as well as potentiating the immune response.

No antibody formation to agarose is detected by a variety of immunological techniques including Ouchterlony, single radial immunodiffusion, “rocket” electrophoresis, dot blots, and immunoperoxidase staining of transfers of isoelectric focusing, two-dimension immunoelectrophoresis, and two-dimension electrophoresis.

When isolated small molecular weight molecules are already available for immunization, electrophoresis is not needed and the small molecules can be added to agarose beads that bind the molecules, and mixed with gelatin as described above. In some cases the small molecular weight molecules can be added to the gelatin directly with having to use and bind them to agarose beads.

The use of coupled small molecular weight antigens is important for antibody production, and a rapid appearance of demonstrable antibody results with small molecules. The minimal amount of small molecular weight molecules that can be used in this procedure is not known, although picomole amounts result in good antibody formation.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

What is claimed is:

1. An immunogenic composition, comprising:
a naturally-chelating nonimmunogenic polymer;
a metal ion; and
an adjuvant,
wherein said metal ion is bound to said naturally-chelating nonimmunogenic polymer.

2. The immunogenic composition of claim 1, wherein said naturally-chelating nonimmunogenic polymer is a polysaccharide.

3. The immunogenic composition of claim 2, wherein said polysaccharide is an alginate.

4. The immunogenic composition of claim 1, wherein said metal ion is selected from the group consisting of: ionic lead, mercury, cadmium, aluminum, lithium, strontium, copper, aluminum, iron, antimony, arsenic, bismuth, chromium, copper, molybdenum, nickel, thallium, technetium, gadolinium, barium, indium, and tin.

5. The immunogenic composition of claim 4, wherein said metal ion is a lead ion.

6. The immunogenic composition of claim 4, wherein said metal ion is a mercury ion.

7. The immunogenic composition of claim 1, wherein said adjuvant is selected from the group consisting of: complete Freund's adjuvant (CFA), incomplete Freund's Adjuvant (IFA), montamide ISA (Incomplete Seppic Adjuvant), Ribi Adjuvant System (RAS); TiterMax; Syntex Adjuvant Formulation (SAF); aluminum salts; nitrocellulose-adsorbed antigen; immune-stimulating complexes (ISCOMs); and Gerbu adjuvant.

8. The immunogenic composition of claim 7, wherein said adjuvant is Complete Freund's adjuvant.

9. The immunogenic composition of claim 7, wherein said adjuvant is aqueous aluminum magnesium hydroxide gel.

10. The immunogenic composition of claim 1, wherein said naturally-chelating nonimmunogenic polymer is formed as a composition of beads.

11. The immunogenic composition of claim 10, wherein said beads have an average diameter of at least about 50 μm.

12. The immunogenic composition of claim 11, wherein said beads have an average diameter of no more than about 250 μm.

13. The immunogenic composition of claim 10, wherein said beads further comprise an agarose.

14. The immunogenic composition of claim 13, wherein said naturally-chelating nonimmunogenic polymer is an alginate.

15. The immunogenic composition of claim 14, wherein said agarose is crosslinked to said alginate.

16. The immunogenic composition of claim 15, wherein said crosslink is formed using divinylsulfone.

17. The immunogenic composition of claim 1, wherein said metal ion is bound saturably to said naturally-chelating nonimmunogenic polymer.

18. The immunogenic composition of claim 17, wherein said metal ion is present in excess of the number of saturable metal ion binding sites of said naturally-chelating nonimmunogenic polymer.

19. The immunogenic composition of claim 1, wherein said metal ion is chelated to said naturally-chelating nonimmunogenic polymer.

20. The immunogenic composition of claim 19, wherein said metal ion is chelated reversibly to said naturally-chelating nonimmunogenic polymer.

21. The immunogenic composition of claim 1, further comprising:
a nonpolysaccharide chelator,
wherein said nonpolysaccharide chelator is capable of chelating said metal ion.

22. The immunogenic composition of claim 21, wherein said nonpolysaccharide chelator is selected from the group consisting of: EDTA, DTPA, meso-2,3-dimercapto succinic acid (DMSA), 2,3-dimercapto-1-propane sulfonate (DMPS), dimercaptopropanol, metallothionein, lactate, penicillamine, deferoxamine, and triethylene tetramine dihydrochloride.

23. The immunogenic composition of claim 22, wherein said nonpolysaccharide chelator is EDTA.

24. The immunogenic composition of claim 21, wherein said chelator is present in an amount sufficient to bind all of said metal ion that is present in excess of metal ion binding sites of said naturally-chelating nonimmunogenic polymer.

25. The immunogenic composition of claim 21, further comprising:
a crosslinked protein,
wherein said protein has been crosslinked in the presence of said naturally-chelating nonimmunogenic polymer and said nonpolysaccharide chelator.

26. The immunogenic composition of claim 25, wherein said protein is selected from the group consisting of: nonxenogeneic serum albumins and gelatins that are liquid at room temperature.

27. The immunogenic composition of claim 26, wherein said protein is a serum albumin.

28. The immunogenic composition of claim 27, wherein said serum albumin is rabbit serum albumin.

29. The immunogenic composition of claim 25, wherein said protein is crosslinked using a crosslinker selected from the group consisting of: formaldehyde, glutaraldehyde, and glyoxal.

30. The immunogenic composition of claim 29, wherein said crosslinker is glutaraldehyde.

31. The immunogenic composition of claim 26, wherein said protein is a gelatin that is liquid at room temperature.

32. The immunogenic composition of claim 31, wherein said gelatin is a cold-water fish gelatin.

33. The immunogenic composition of claim 25, wherein said protein is present in an amount sufficient, upon crosslinking, to create a gel at room temperature.

34. The immunogenic composition of claim 1, wherein said composition is particulate.

35. The immunogenic composition of claim 34, wherein said metal ion-bound naturally-chelating nonimmunogenic polymer is substantially dried before admixture with said adjuvant.

36. The immunogenic composition of claim 35, wherein said metal ion-bound naturally-chelating nonimmunogenic polymer is lyophilized before admixture with said adjuvant.

37. An immunogenic composition, comprising:
a particulate naturally-chelating nonimmunogenic polymer complexed with a metal ion, and an adjuvant.

38. The immunogenic composition of claim 37, wherein said particulate, metal ion-complexed, naturally-chelating nonimmunogenic polymer is depotized.

39. The immunogenic composition of claim 38, wherein the metal ion of said depotized particulate metal ion-complexed naturally-chelating nonimmunogenic polymer is dynamically bound by a plurality of chelators.

40. An immunogenic composition, comprising:
an immunogen; and
a crosslinked gelatin soluble at room temperature,
wherein said gelatin is crosslinked in the presence of said immunogen.

41. The immunogenic composition of claim 40, wherein said gelatin is a cold-water fish gelatin.

42. The immunogenic composition of claim 40, further comprising an adjuvant.

43. A method of making an antibody reagent, the method comprising:
immunizing a nonhuman animal with the immunogenic composition of any one of claims 1, 37 or 40; and then
isolating an antibody that binds to said immunogenic composition.

44. The method of claim 43, wherein said isolating comprises the step of:
isolating said antibody from the serum of said nonhuman animal.

45. The method of claim 43, wherein said isolating comprises the initial step of:
isolating a cell that secretes an antibody that binds to said immunogenic composition.

46. The method of claim 45, wherein said cell is a clonal cell line.

47. The method of claim 44, further comprising:
affinity purifying said antibody reagent.

48. The method of claim 45, further comprising:
affinity purifying said antibody reagent.

49. An antibody reagent, comprising:
at least one isolated antibody,
wherein said antibody is specific for a metal ion as complexed to a naturally-chelating nonimmunogenic polymer.

50. The antibody reagent of claim 49, wherein said naturally-chelating nonimmunogenic polymer is an alginate.

51. The antibody reagent of claim 50, wherein said metal ion is selected from the group consisting of: ionic lead, mercury, cadmium, aluminum, lithium, strontium, copper, aluminum, iron, antimony, arsenic, bismuth, chromium, copper, molybdenum, nickel, thallium, technetium, gadolinium, barium, indium, and tin.

52. The antibody reagent of claim 51, wherein said metal ion is a lead ion.

53. The antibody reagent of claim 51, wherein said metal ion is a mercury ion.
